(19) United States
(12) Patent Application Publication
(10) Pub. No.: US 2024/0197206 A1
(43) Pub. Date: Jun. 20, 2024

(54) INFORMATION PROCESSING APPARATUS, INFORMATION PROCESSING METHOD, AND COMPUTER-READABLE MEDIUM

(71) Applicant: NEC Corporation, Minato-ku, Tokyo (JP)

(72) Inventors: Hiroshi Kajitani, Tokyo (JP); Eisuke Orito, Tokyo (JP); Kenichiro Fukushi, Tokyo (JP); Chenhui Huang, Tokyo (JP); Fumiyuki Nihey, Tokyo (JP)

(73) Assignee: NEC Corporation, Minato-ku, Tokyo (JP)

(21) Appl. No.: 18/287,421

(22) PCT Filed: Apr. 30, 2021
(86) PCT No.: PCT/JP2021/017158

§ 371 Date: Oct. 18, 2023

(51) Int. Cl. A61B 5/11; A61B 5/00

(52) U.S. Cl. CPC A61B 5/112; A61B 5/1121; A61B 5/6807; A61B 5/7221; A61B 5/7285; A61B 2562/0219

(57) ABSTRACT

An information processing apparatus (10) includes an acquisition means (11) for acquiring information based on a sensor (21) attached to a foot of a user, a detection means (12) for detecting, when a gait of the user cannot be detected based on the information indicating an angle between a sole of the foot of the user and the ground acquired by the acquisition means and a first threshold value, the gait of the user based on the information indicating the angle and a second threshold value smaller than the first threshold value, and an output means (13) for outputting information based on a result of the detection performed by the detection means.

### TECHNICAL FIELD

The present disclosure relates to an information processing apparatus, an information processing method, and a non-temporary computer readable medium storing a program.

### BACKGROUND ART

Techniques for detecting the gait of a user (a walking pattern) based on data measured by a sensor are known (see, for example, Patent Literatures 1 and 2).

### CITATION LIST

### Patent Literature

Patent Literature 1: Japanese Unexamined Patent Application Publication No. 2016-042879
Patent Literature 2: Japanese Unexamined Patent Application Publication No. 2005-342254

### SUMMARY OF INVENTION

### Technical Problem

However, with the techniques described in Patent Literatures 1 and 2, there is such a problem that information on the gait of the user may not be able to be appropriately detected.

An object of the present disclosure is to provide, in view of the above-mentioned problem, an information processing apparatus, an information processing method, and a non-temporary computer-readable medium storing a program each adapted to detect information on the gait of a user.

### Solution to Problem

According to a first aspect of the present disclosure an information processing apparatus includes: acquisition means for acquiring information based on a sensor attached to a foot of a user;

detection means for detecting, when a gait of the user cannot be detected based on the information indicating an angle between a sole of the foot of the user and the ground acquired by the acquisition means and a first threshold value, the gait of the user based on the information indicating the angle and a second threshold value smaller than the first threshold value; and output means for outputting information based on a result of the detection performed by the detection means.

According to a second aspect of the present disclosure, an information processing method includes: acquiring information based on a sensor attached to a foot of a user; detecting, when a gait of the user cannot be detected based on the acquired information indicating an angle between a sole of the foot of the user and the ground and a first threshold value, the gait of the user based on the information indicating the angle and a second threshold value lower than the first threshold value; and outputting information based on a result of the detection.

According to a third aspect of the present disclosure, a non-transitory computer-readable medium stores a program for causing an information processing apparatus to perform processes of: acquiring information based on a sensor attached to a foot of a user; detecting, when a gait of the user cannot be detected based on the acquired information indicating an angle between a sole of the foot of the user and the ground and a first threshold value, the gait of the user based on the information indicating the angle and a second threshold value smaller than the first threshold value; and outputting information based on a result of the detection.

### Advantageous Effects of Invention

According to one aspect, information on the gait of a user can be appropriately detected.

### BRIEF DESCRIPTION OF DRAWINGS

FIG. 1 is a diagram showing an example of a configuration of an information processing apparatus for performing a generation process according to an example embodiment;

FIG. 2 is a diagram showing an example of a configuration of an information processing system according to an example embodiment;

FIG. 3 is a diagram showing an example of a hardware configuration of an information processing apparatus according to an example embodiment;

FIG. 4 is a diagram showing an example of a position where a sensor according to an example embodiment is attached;

FIG. 5 is a diagram showing an example of a configuration of a measuring apparatus according to an example embodiment;

FIG. 6 is a flowchart showing an example of a detection process according to an example embodiment;

FIG. 7 is a flowchart showing an example of a detection process according to an example embodiment;

FIG. 8 is a diagram showing an example of a transition of pitch and roll at each point as a healthy subject walks, as measured by a sensor according to an example embodiment;

FIG. 9 is a diagram showing an example of a transition of pitch and roll in each point as a patient walks, as measured by a sensor according to an example embodiment;

FIG. 10 is a diagram showing an example of transition of acceleration at each point as a healthy subject walks, as measured by a sensor according to an example embodiment;

FIG. 11 is a diagram showing an example of transition in acceleration at each point as a patient walks, as measured by a sensor according to an example embodiment;

FIG. 12 is a diagram showing an example of transition in angular velocity in each point as a healthy subject walks, as measured by a sensor according to an example embodiment; and

FIG. 13 is a diagram showing an example of transition in angular velocity in each point as a patient walks, as measured by a sensor according to an example embodiment.

### EXAMPLE EMBODIMENT

The principles of the present disclosure will be described with reference to some illustrative exemplary example embodiments. It should be understood that these example embodiments are described for illustrative purposes only and will assist those skilled in the art in understanding and implementing the present disclosure without suggesting any limitations with respect to the scope of the present disclosure. The disclosures set forth herein may be implemented in a variety of ways other than those described below.

In the following description and claims, unless otherwise defined, all technical and scientific terms used herein have the same meanings as are generally understood by those skilled in the art to which the present disclosure pertains.

Hereinafter, example embodiments of the present disclosure will be described with reference to the drawings.

### First Example Embodiment

### <Configuration>

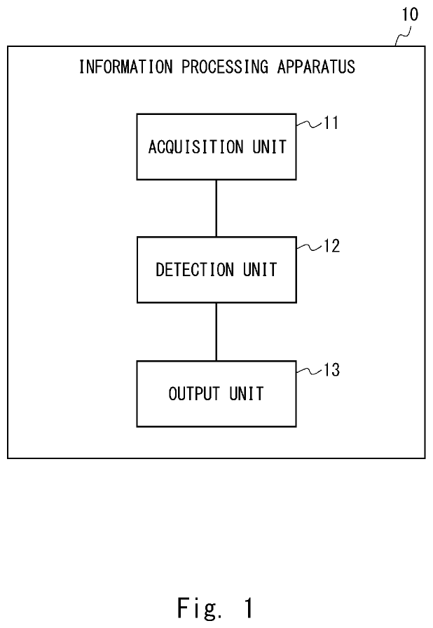

A configuration of an information processing apparatus 10 according to an example embodiment will be described with reference to FIG. 1. FIG. 1 is a diagram showing an example of the configuration of the information processing apparatus 10 according to an example embodiment. The information processing apparatus 10 includes an acquisition unit 11, a detection unit 12, and an output unit 13. These components may be implemented by one or more programs installed in the information processing apparatus 10 in conjunction with hardware such as a processor 101 and a memory 102 of the information processing apparatus 10.

The acquisition unit 11 acquires various kinds of information from a storage part inside the information processing apparatus 10 or an external device. For example, the acquisition unit 11 acquires information indicating an angle between the sole of the foot (sole) and the ground in a direction in which the user walks measured by a sensor attached to the foot of the user (hereinafter also referred to as a “sole angle” as appropriate).

When the detection unit 12 cannot detect the gait of the user based on the information acquired by the acquisition unit 11 and a first threshold value, the detection unit 12 detects the gait of the user gait based on the information acquired by the acquisition unit 11 and a second threshold value smaller than the first threshold value.

The output unit 13 outputs (sends, records) various kinds of information to a storage unit inside the information processing apparatus 10 or to an external device. The output unit 13 outputs, for example, information based on the result of detection performed by the detection unit 12.

### Second Example Embodiment

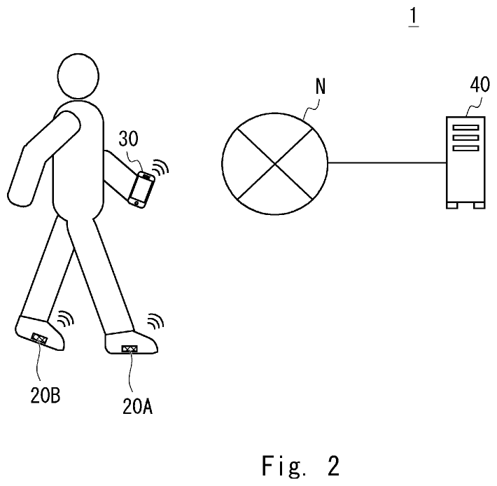

Next, with reference to FIG. 2, the configuration of an information processing system 1 according to an example embodiment will be described.

### <System Configuration>

FIG. 2 is a diagram showing an example of a configuration of the information processing system 1 according to an example embodiment. In the example shown in FIG. 2, the information processing system 1 includes a measuring apparatus 20A and a measuring apparatus 20B (hereinafter, when no distinction is necessary, it is also referred to simply as a “measuring apparatus 20”). The information processing system 1 also includes a user terminal 30 and a server 40. The numbers of the measuring apparatuses 20, the user terminals 30, and the servers 40 are not limited to the example shown in FIG. 2. The measuring apparatus 20, the user terminal 30, and the server 40 are examples of the information processing apparatus 10, respectively.

In the following, an example in which detection is performed by the measuring apparatus 20 will be described as an example. The detection process may be executed by at least one of the measuring apparatus 20, the user terminal 30, and the server 40.

The measuring apparatus 20 and the user terminal 30 may be connected in such a way that they can communicate with each other through, for example, short-range wireless communication such as BLE (Bluetooth (registered trademark) Low Energy) or a cable.

In the example shown in FIG. 2, the user terminal 30 and the server 40 are connected in such a way that they can communicate with each other through a network N. Examples of the network N include, for example, the Internet, a mobile communication system, a wireless LAN (Local Area Network), a short-range wireless communication such as BLE, a LAN, and a bus. Examples of the mobile communication system include, for example, a fifth-generation mobile communication system (5G), a fourth-generation mobile communication system (4G), a third-generation mobile communication system (3G), and the like.

The measuring apparatus 20 has a sensor 21 attached to the foot of a user. The measuring apparatus 20 outputs data measured using the sensor 21 to an external device such as the user terminal 30 or the server 40. The measuring apparatus 20 may transmit data to the server 40 without having to use the user terminal 30.

The user terminal 30 may be a device such as, for example, a smartphone, a tablet, a personal computer, an Internet of Things (IOT) communication apparatus, and a cellular phone. The user terminal 30 transmits data acquired from, for example, the measuring apparatus 20 to the server 40. The user terminal 30 displays information on the gait of the user on the screen based on, for example, data measured by the sensor 21.

The server 40 is, for example, a server, a cloud computing system, a personal computer, a smartphone, or the like. The server 40 records data measured by the sensor 21, for example, and displays information on the gait of the user on the user terminal 30 based on the recorded data.

### <Hardware Configuration>

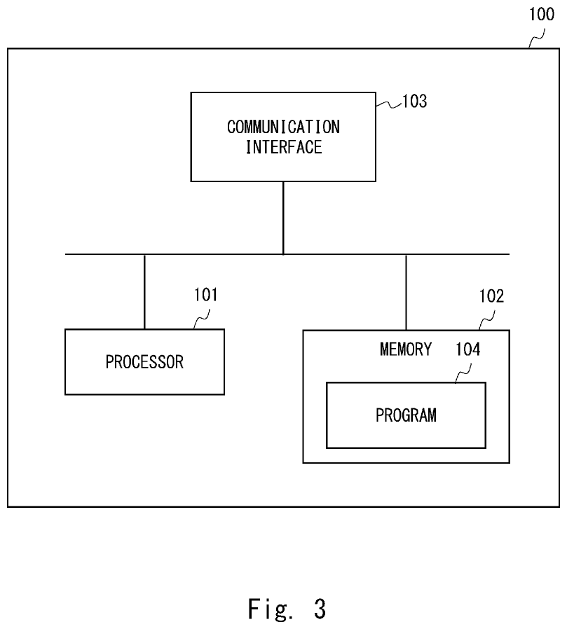

FIG. 3 is a diagram showing an example of a hardware configuration of the information processing apparatus 10 according to an example embodiment. In the example of FIG. 3, the information processing apparatus 10 (a computer 100) includes the processor 101, the memory 102, and a communication interface 103. These parts may be connected to one another by a bus or the like. The memory 102 stores at least a part of a program 104. The communication interface 103 includes interfaces required for communication with other network elements.

When the program 104 is executed in conjunction with the processor 101, the memory 102, and the like, at least a part of the processing of the example embodiment of the present disclosure is performed by the computer 100. The memory 102 may be of any type suitable for a local area network. The memory 102 may, as a non-limiting example, be a non-temporary computer readable storage medium. The memory 102 may also be implemented using any suitable data storage technology, such as semiconductor-based memory devices, magnetic memory devices and systems, optical memory devices and systems, and fixed and removable memories. Although only one memory 102 is shown for the computer 100, the computer 100 may have several physically distinct memory modules. The processor 101 may be of any type. The processor 101 may include one or more of a general-purpose computer, a dedicated computer, a microprocessor, a digital signal processor (DSP), and, as a non-limiting example, a processor based on a multi-core processor architecture. The computer 100 may include a plurality of processors such as an application-specific integrated circuit chip that is temporally dependent on a clock that synchronizes the main processor.

The example embodiments of the present disclosure may be implemented in hardware or dedicated circuits, software, logic or any combination thereof. Some aspects may be implemented in hardware, while others may be implemented in firmware or software that may be executed by a controller, a microprocessor or other computing devices.

The present disclosure also provides at least one computer program product that is tangibly stored on a non-temporary computer readable storage medium. The computer program product includes computer executable instructions, such as instructions contained in program modules, and is executed on a device on a target real or virtual processor to execute the process or the method of the present disclosure. A program module includes routines, programs, libraries, objects, classes, components, data structures, and the like that perform a specific task or implement a specific abstract data type. The functions of a program module may be combined or divided among the program modules as desired in various example embodiments. The machine executable instructions in a program module can be executed locally or within a distributed device. In a distributed device, program modules can be arranged on both local and remote storage media.

The program codes for implementing the methods of the disclosure may be written in any combination of one or more programming languages. These program codes may be provided to a processor or controller of a general-purpose computer, a dedicated computer, or other programmable data processing device. When the program codes are executed by a processor or a controller, the functions/operations in the flowchart and/or implemented block diagram are executed. The program codes are executed entirely on the machine, partly on the machine, as a standalone software package, partly on the machine, partly on a remote machine, or entirely on a remote machine or a server.

The program may be stored and provided to a computer using various types of non-temporary computer-readable media. Non-temporary computer-readable media include various types of tangible recording media. Examples of non-temporary computer-readable media include magnetic recording media, magneto-optical recording media, optical disk media, semiconductor memory, and the like. Magnetic recording media include, for example, flexible disks, magnetic tapes, hard disk drives, and the like. Magneto-optical recording media include, for example, magneto-optical disks and the like. Optical disk media include, for example, Blu-ray disks, compact disc (CD)-ROM (Read Only Memory), CD-R (Recordable), CD-RW (ReWritable) and the like. Semiconductor memory includes, for example, solid-state drives, mask ROM, PROM (Programmable ROM), EPROM (Erasable PROM), flash ROM, RAM (random access memory) and the like. Programs may also be provided to the computer by various types of temporary computer readable media. Examples of temporary computer readable media include electrical signals, optical signals, and electromagnetic waves. Temporary computer readable media can provide programs to computers via wired or wireless communication channels, such as wires and optical fibers.

### <Regarding Measuring Apparatus 20>

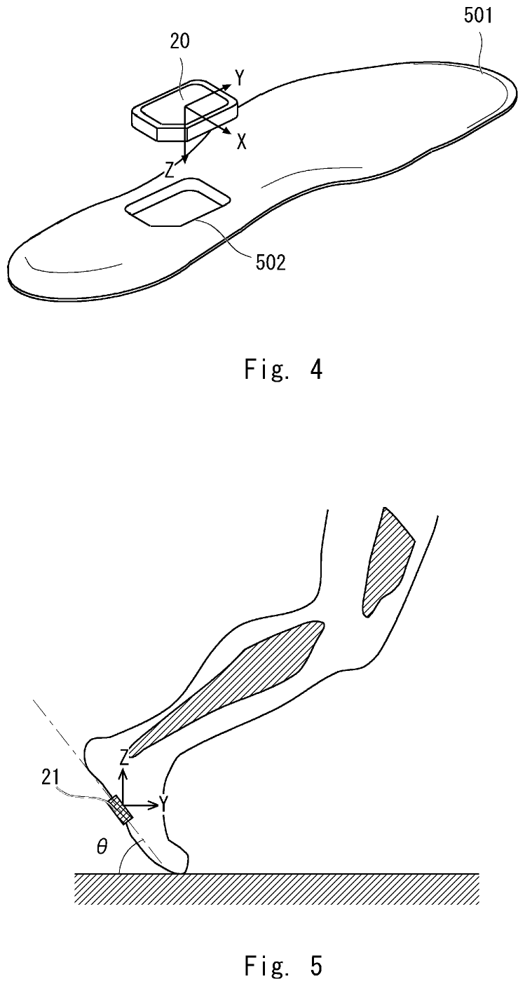

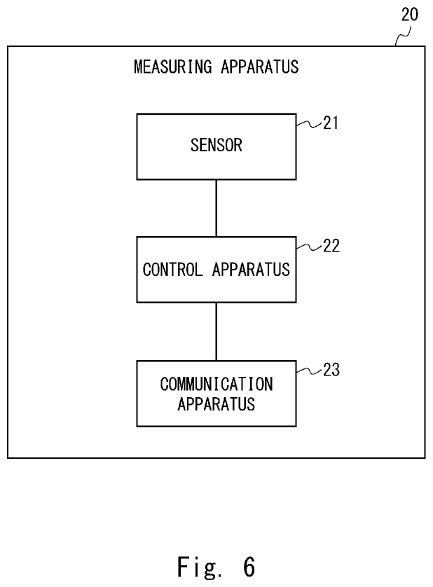

Next, with reference to FIGS. 4 to 6, an example of the measuring apparatus 20 according to an example embodiment will be described. FIG. 4 is a diagram showing an example of a position where the sensor 21 according to an example embodiment is attached. FIG. 5 is a diagram showing an example of data measured by the sensor 21 according to an example embodiment. FIG. 6 is a diagram showing an example of a configuration of the measuring apparatus 20 according to an example embodiment.

In the example shown in FIG. 4, the measuring apparatus 20 is accommodated (installed) in an arch 502 of an insole (insole) 501 of a shoe worn by a user. It should be noted that the sensor 21 of the measuring apparatus 20 may be attached to the user on the sole side of the foot of the user, between the arch and the heel of the foot of the user. As shown in FIGS. 4 and 5, the sensor 21 may measure (perform calculation and measurement), for example, acceleration in the direction in which the user walks (Y direction), acceleration in the vertical upward direction (Z direction), and acceleration in the direction of the user's other foot (X direction) perpendicular to the direction in which the user walks and the vertical direction. Further, as shown in FIG. 5, a sole angle θ, for example, may be measured by the sensor 21.

In the example shown in FIG. 6, the measuring apparatus 20 includes the sensor 21, a control apparatus 22, and a communication apparatus 23. The sensor 21 measures, for example, acceleration and angular velocity. The sensor 21 may be, for example, an inertial measurement unit (IMU) having a 3-axis acceleration sensor and a 3-axis gyro sensor. The control apparatus 22 outputs data measured using the sensor 21 to an external device using the communication apparatus 23. The control apparatus 22 may have the same configuration as the computer 100 shown in FIG. 3. In this case, the control apparatus 22 may be, for example, a microcontroller or the like.

### <Processing>

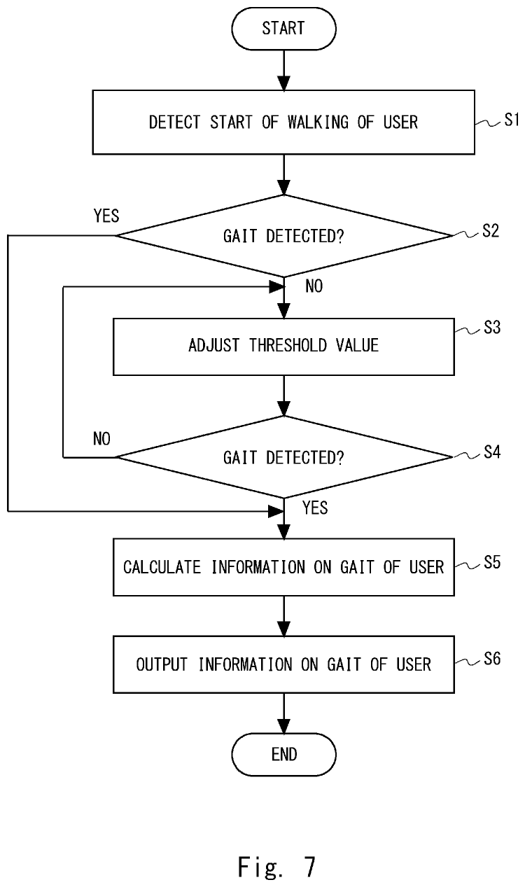

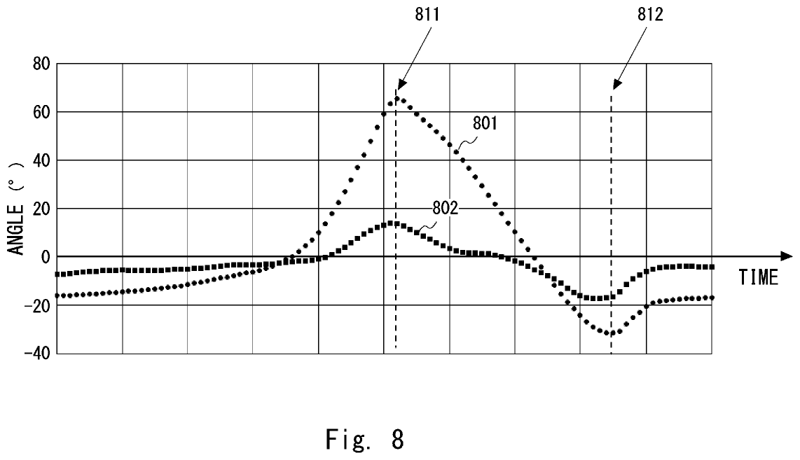

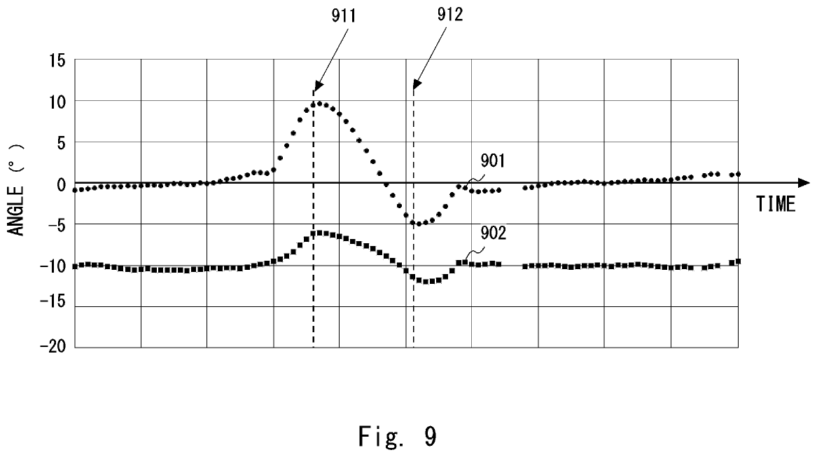

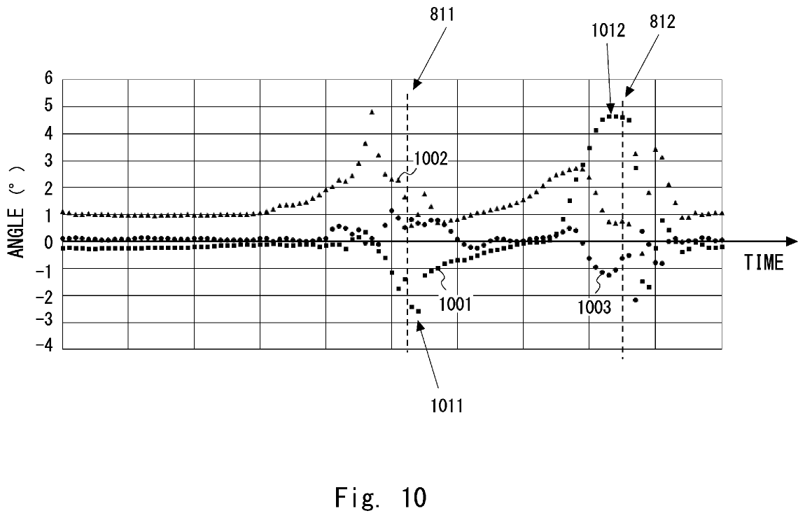

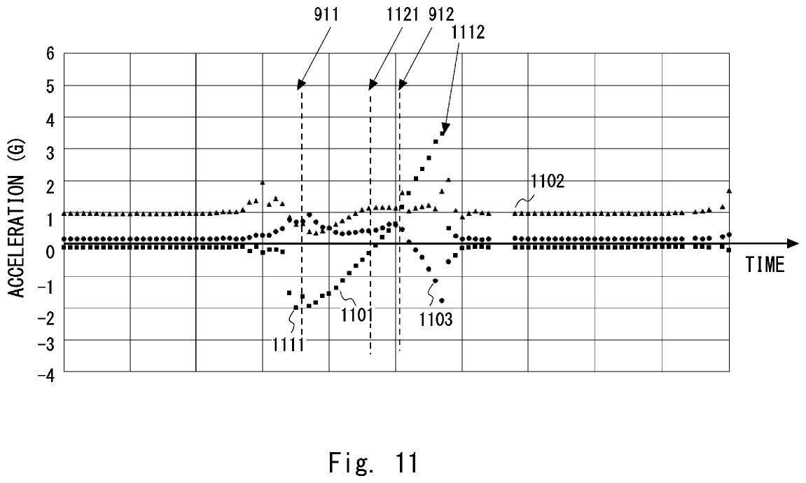

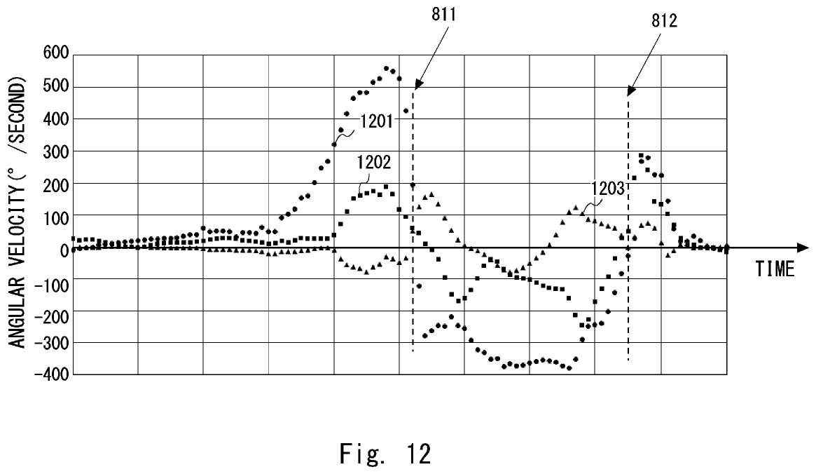

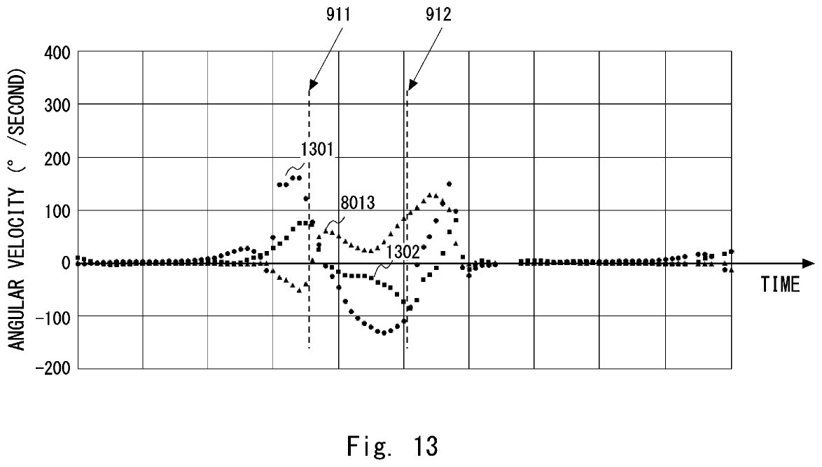

Next, with reference to FIGS. 7 to 13, an example of the detection processing according to an example embodiment will be described. FIG. 7 is a flowchart showing an example of the detection processing according to an example embodiment. FIG. 8 is a diagram showing an example of a transition of pitch and roll in each point as a healthy subject walks, as measured by the sensor 21 according to an example embodiment. FIG. 9 is a diagram showing an example of a transition of pitch and roll in each point as a patient walks, as measured by the sensor 21 according to an example embodiment. FIG. 10 is a diagram showing an example of transition of acceleration at each point as a healthy subject walks, as measured by the sensor 21 according to an example embodiment. FIG. 11 is a diagram showing an example of transition of acceleration at each point when a patient walks, as measured by the sensor 21 according to an example embodiment. FIG. 12 is a diagram showing an example of transition of angular velocity at each point as a healthy subject walks, as measured by the sensor 21 according to an example embodiment. FIG. 13 is a diagram showing an example of transition in angular velocity in each point as a patient walks, as measured by the sensor 21 according to an example embodiment.

In Step S1, the detection unit 12 detects that the user wearing the sensor 21 has started walking. Here, the detection unit 12 may, for example, determine that the user has started walking upon reception of a predetermined command from an external device. In this case, for example, the detection unit 12 of the measuring apparatus 20 may receive the command from the user terminal 30, which has accepted the operation from the user. Thus, for example, the measurement of the gait of the user can be started in response to an operation performed by a doctor at a hospital or the like.

In addition, the detection unit 12 may determine that the user has started walking when at least one of the acceleration and the angular velocity measured by the sensor 21 is equal to or greater than the threshold value. Thus, for example, the burden imposed on the user of having to perform an operation can be reduced.

Subsequently, the detection unit 12 determines whether or not the gait of the user can be detected based on the predetermined threshold value and the information measured by the sensor 21 (Step S2). Here, the detection unit 12 may determine, for example, whether or not the time length during which the user takes one step forward (for example, from lifting one foot off the ground to lowering it back to the ground) can be measured based on the sole angle measured by the sensor 21. In this case, the detection unit 12 may determine that the time length for the user take one step forward can be measured if, for example, the local maximum value (the maximum value) of the sole angle in the specified period is equal to or greater than the threshold value for the first local maximum value (an example of the “first threshold value”, e.g., 45°). In this case, the detection unit 12 may calculate (determine) the time length for the user take one step forward based on the time length between each point at which the angle has the local maximum value.

Further, the detection unit 12 may determine that the time length for the user take one step forward can be measured, if, for example, the local minimum value (the minimum value) of the sole angle in the specified period is equal to or greater than the threshold value for the first local minimum value (an example of “first threshold value”, e.g.,)−20°. In this case, the detection unit 12 may calculate (determine) the time length for the user take one step forward based on the time length between each point at which the angle has the minimum value.

FIG. 8 shows an example of transition 801 of a sole angle (pitch) and transition 802 of a rotation angle (roll) with respect to the direction in which the user walks at each point as a healthy subject walks. In the example shown in FIG. 8, a sole angle has a maximum local value (about 65°) at point 811 and a minimum local value (about)−30° at point 812.

FIG. 9 shows an example of transition 901 of a sole angle (pitch) and transition 902 of a rotation angle (roll) with respect to the direction in which the user walks at each point when a patient with a foot disorder or the like walks. In the example shown in FIG. 9, a sole angle has a local maximum value (about 10°) at point 911 and a minimum value (about)−5° at point 912. Therefore, in the case a sole angle transits as shown in FIG. 9, it is determined that the detection unit 12 cannot detect the gait of the user.

If the gait of the user can be detected (YES in Step S2), the process proceeds to Step S5. On the other hand, if the gait of the user cannot be detected (NO in Step S2), the detection unit 12 adjusts the threshold value and the like (Step S3). Thus, the gait of the user can be appropriately detected even when the fluctuation in the user's sole angle is smaller than that of a healthy subject due to, for example, a foot injury, light-headedness due to a disease affection, or decline in the leg and hip function due to aging. Here, the detection unit 12 updates the threshold value to a value that is more moderate than the current threshold value. In this case, the detection unit 12 may use the value obtained by multiplying the current threshold value by a predetermined coefficient (e.g., 0.8) as the threshold value in the subsequent processing.

Further, the detection unit 12 may also determine the second threshold value based on the attributes of the user who is wearing the sensor 21. Thus, for example, faster adjustment of the threshold value for detecting the gait of the user can be realized. In this case, the detection unit 12 of the measuring apparatus 20 may receive information indicating the attributes of the user specified by the user from the user terminal 30. The detection unit 12 may set, as an initial value of the second threshold value, a value previously set (registered) so as to correspond to the attributes of the user specified by the user, including sex, age, the extent of a foot injury, the degree of a disease, etc.

The detection unit 12 may also determine a second threshold value based on at least one of the acceleration and the angular velocity measured by the sensor 21. Thus, for example, faster adjustment of the threshold value for detecting the gait of the user can be realized. In this case, the detection unit 12 may set, for example, the values previously set so as to correspond to the maximum and the minimum values of acceleration in the direction in which the user walks and the maximum and the minimum values of the sole angle as the initial value of the second threshold value.

The detection unit 12 may also determine the second threshold value based on at least one of the heart rate and the skin temperature of the user wearing the sensor 21. Thus, for example, the gait of the user can be detected more appropriately in accordance with the emotional state of the user. In this case, for example, the acquisition unit 11 may acquire information indicating the user's emotions estimated based on the heart rate and the skin temperature measured by a wearable device or the like worn by the user. The detection unit 12 may set a value previously set (registered) so as to correspond to the user's emotions as the initial value of the second threshold value. The detection unit 12 may record the value of the second threshold value for each user's emotions, the second threshold value being adjusted so that the gait of the user can be detected. The detection unit 12 may then measure, using a second threshold value corresponding to the current emotion of the user, the length of time required for the current user to take one step forward.

Subsequently, the detection unit 12 determines whether or not the gait of the user can be detected based on the adjusted threshold value (the second threshold value) and the like and the information measured by the sensor 21 (Step S4). Here, the detection unit 12 may increase the sampling frequency (the sampling rate) of the analog signal indicating the sole angle measured by the sensor 21 from the first sampling frequency (e.g., 100 Hz) for a healthy subject to the second sampling frequency (e.g., 150 Hz) for a healthy subject. The detection unit 12 may then measure the length of time required for the user to take one step forward based on the sole angle sampled at the second sampling frequency and the second threshold value. Thus, the length of time required for the user to take one step forward can be more appropriately measured in the caser where, for example, one stride is relatively short and the length of time required for the user to take one step forward is relatively short because the user has a foot disorder or the like.

The detection unit 12 may also measure the length of time required for the user to take one step forward based on the sole angle, the second threshold value, and acceleration in the direction in which the user walks measured by the sensor 21. Thus, the length of time required for the user to take one step can be more appropriately measured even in the case where, for example, the gait of the user cannot be appropriately detected based on acceleration in the vertical upward direction measured by the sensor 21 due to the high impact absorption of the soles and insole of the shoes worn by the user. In this case, the detection unit 12 may, for example, first calculate a representative value (e.g., mean, median, mode) of the length of time between each point at which the sign of acceleration in the direction in which the user walks measured by the sensor 21 changes from positive to negative. The detection unit 12 may calculate the representative value as the length of time required for the user to take one step forward when there is one or more point at which the extreme value of the sole angle is equal to or greater than the second threshold value in the length of time of the representative value.

FIG. 10 shows an example of transition 1001 of acceleration in a direction opposite to the direction of walking (−Y direction), transition 1002 of acceleration in a vertical upward direction (Z direction), and transition 1003 of acceleration in an X direction at the same points as those when a healthy subject walks shown FIG. 8. In the example of FIG. 10, acceleration is in the −Y direction (the reverse of the sign of the Y direction). Therefore, the local maximum value of acceleration in the direction of walking (Y direction) is about 2.8G, which is obtained by reversing the sign of the value 1011, and the local minimum value is about −4.8G, which is obtained by reversing the sign of the value 1012. In the example shown in FIG. 10, the point at which acceleration in the Y direction has the local maximum value and the point 811 at which the sole angle has the local maximum value generally coincide with each other, and the point at which acceleration in the Y direction has the local minimum value and the point 812 where the sole angle has the local minimum value also generally coincide with each other.

In addition, FIG. 11 shows an example of transition 1101 in acceleration in the direction opposite to the direction of walking (−Y direction), transition 1102 in acceleration in the vertical upward direction (Z direction), and transition 1103 in acceleration in the X direction in the same points as those shown in FIG. 9 when a patient with a foot disorder or the like walks. In the example shown FIG. 11, acceleration is in the −Y direction (the reverse of the sign of the Y direction). Therefore, the local maximum value of acceleration in the direction of walking (Y direction) is about 2G, which is obtained by reversing the sign of the value 1111, and the local minimum value is about −3.5G, which is obtained by reversing the sign of the value 1112. In the example shown in FIG. 11, at point 1121, the sign of acceleration in the Y direction changes from positive to negative. In the example shown in FIG. 11, the point at which acceleration in the Y direction has the local maximum value and the point 911 at which the sole angle has the local maximum value generally coincide with each other. On the other hand, the point at which acceleration in the Y direction has the local minimum value and the point 912 at which the sole angle has the local minimum value do not coincide with each other. Therefore, the detection unit 12 can more appropriately measure the time length for the user to take one step forward by using, for example, information such as the point at which the sign of acceleration in the Y direction changes from positive to negative.

The detection unit 12 may also measure the time length for the user take one step forward based on the sole angle, the second threshold value, and the angular velocity of the sole angle. Thus, the time length for the user takes one step forward can be more appropriately measured, even in the case where, for example, the peak of sole angle is smooth and there is a variation in point that becomes the extreme value in each step movement. In this case, the detection unit 12 may, for example, determine that point at which the sign of angular velocity of the user's sole angle measured by the sensor 21 changes from positive to negative to be the point at which the sole angle has the local maximum value. The detection unit 12 may, for example, determine that point at which the sign of the user's sole angle angular velocity measured by the sensor 21 changes from negative to positive is the point at which the sole angle has the local minimum value.

FIG. 12 shows an example of transition 1201 in the angular velocity of the sole angle (pitch), transition 1202 of the angular velocity of roll, and transition 1203 of the angular velocity of Yaw at each point shown in FIG. 8 when a healthy subject walks. In the example shown in FIG. 12, the point 811 at which the sign of angular velocity of the user's sole angle changes from positive to negative and the point at which the sole angle has the local maximum value generally coincide with each other, and the point 812 at which the sign of angular velocity of the user's sole angle changes from negative to positive and the point at which the sole angle has the local minimum value also generally coincides with each other.

FIG. 13 shows an example of transition 1301 in the angular velocity of the sole angle (pitch), transition 1302 of angular velocity of roll, and transition 1303 of the angular velocity of Yaw at the same point as those show in FIG. 9 when a patient with a foot disorder or the like walks. In the example shown in FIG. 13, the point 911 at which the sign of angular velocity of the user's sole angle changes from positive to negative and the point at which the sole angle has the local maximum value generally coincide with each other, and the point 912 at which the sign of angular velocity of the user's sole angle changes from negative to positive and the point at which the sole has the local minimum value also generally coincides with each other.

If the gait of the user cannot be detected (NO in Step S4), the process proceeds to Step S3. On the other hand, if the gait of the user can be detected (YES in Step S4), the detection unit 12 calculates information on the gait of the user (Step S5). Here, the detection unit 12 may measure, for example, the time length while the user takes one step forward (for example, between lifting one foot off the ground and lowering it back to the ground) based on sole angle measured by the sensor 21. Then, the detection unit 12 may calculate the walking speed, the stride, foot lifted height, outside turning distance, etc. based on acceleration measured by the sensor 21 in the time length for the user to take one step forward. The detection unit 12 may also calculate the ground contact angle and the ground rise-off angle of the user's feet based on the angular velocity measured by the sensor 21 in the time length for the user to take one step forward. The ground contact angle may be the angle between the sole of the foot and the ground when the foot touches the ground in the direction in which the user walks. The ground rise-off angle may be the angle between the sole of the foot and the ground when the foot is lifted off the ground in the direction in which the user walks.

Subsequently, the output unit 13 outputs the information on the gait of the user calculated by the detection unit 12 (Step S6), and the process ends. Thus, for example, the server 40 can transmit advice for improving the gait of the user, video for performing walking training, etc. to the user terminal 30.

### Modified Example

The information processing apparatus 10 may be a device included in one housing, but the information processing apparatus 10 of the present disclosure is not limited thereto. Each part of the information processing apparatus 10 may be implemented by, for example, cloud computing configured of, for example, one or more computers. The parts of the information processing apparatus 10 may also be implemented, for example, by a plurality of devices among the measuring apparatus 20, the user terminal 30, and the server 40. Such the information processing apparatus 10 is also included in an example of “information processing apparatus” in the present disclosure.

It should be noted that present disclosure is not limited to the above embodiment, and can be appropriately changed to the extent that it does not deviate from the purpose.

A part or all of the above example embodiments may also be described as in the following supplementary notes, but not limited to the following.

(Supplementary note 1)

An information processing apparatus comprising:
acquisition means for acquiring information based on a sensor attached to a foot of a user;
detection means for detecting, when a gait of the user cannot be detected based on the information indicating an angle between a sole of the foot of the user and the ground acquired by the acquisition means and a first threshold value, the gait of the user based on the information indicating the angle and a second threshold value smaller than the first threshold value; and
output means for outputting information based on a result of the detection performed by the detection means.

### (Supplementary Note 2)

The information processing apparatus described in Supplementary Note 1, wherein the acquisition means acquires the information based on the sensor attached to the user at any position between the arch and the heel of the foot of the user.

### (Supplementary Note 3)

The information processing apparatus described in Supplementary Note 1 or 2, wherein the detection means detects, in the case where the gait of the user cannot be detected based on the first threshold value when a predetermined command is received from an external device, the gait of the user based on the second threshold value.

### (Supplementary Note 4)

The information processing apparatus described in any one of Supplementary Notes 1 to 3, wherein the detection means detects, in the case where at least one of acceleration and angular velocity measured by the sensor is equal to or greater than a threshold value and the gait of the user cannot be detected based on the first threshold value, the gait of the user based on the second threshold value.

### (Supplementary Note 5)

The information processing apparatus described in any one of Supplementary Notes 1 to 4, wherein the detection means detects, in the case where the gait of the user cannot be detected based on the information indicating the angle sampled at a first sampling frequency and the first threshold value, the gait of the user based on the information indicating the angle sampled by a second sampling frequency higher than the first sampling frequency.

### (Supplementary Note 6)

The information processing apparatus described in any one of Supplementary Notes 1 to 5, wherein the detection means detects, in the case where the gait of the user cannot be detected based on the first threshold value, the gait of the user based on the information indicating the angle, the second threshold value, and the acceleration in a direction in which the user walks measured by the sensor.

### (Supplementary Note 7)

The information processing apparatus described in any one of Supplementary Notes 1 to 6, wherein the detection means determines the second threshold value based on attributes of the user.

### (Supplementary Note 8)

The information processing apparatus described in any one of Supplementary Notes 1 to 7, wherein the detection means determines the second threshold value based on at least one of the acceleration and the angular velocity measured by the sensor.

### (Supplementary Note 9)

The information processing apparatus described in any one of Supplementary Notes 1 to 8, wherein the detection means determines the second threshold value based on at least one of a heart rate and a skin temperature of the user.

### (Supplementary Note 10)

An information processing method comprising:
acquiring information based on a sensor attached to a foot of a user;
detecting, when a gait of the user cannot be detected based on the acquired information indicating an angle between a sole of the foot of the user and the ground and a first threshold value, the gait of the user based on the information indicating the angle and a second threshold value lower than the first threshold value; and
outputting information based on a result of the detection.

### (Supplementary Note 11)

A non-transitory computer-readable medium storing a program for causing an information processing apparatus to perform processes of:
acquiring information based on a sensor attached to a foot of a user;
detecting, when a gait of the user cannot be detected based on the acquired information indicating an angle between a sole of the foot of the user and the ground and a first threshold value, the gait of the user based on the information indicating the angle and a second threshold value smaller than the first threshold value; and
outputting information based on a result of the detection.

### REFERENCE SIGNS LIST

1 INFORMATION PROCESSING SYSTEM
10 INFORMATION PROCESSING APPARATUS
11 ACQUISITION UNIT
12 DETECTION UNIT
13 OUTPUT UNIT
20 MEASURING APPARATUS
21 SENSOR
22 CONTROL APPARATUS
23 COMMUNICATION APPARATUS
30 USER TERMINAL
40 SERVER

What is claimed is:

1. An information processing apparatus comprising:
at least one memory storing instructions, and
at least one processor configured to execute the instructions to;
acquire information based on a sensor attached to a foot of a user;
detect, when a gait of the user cannot be detected based on the information indicating an angle between a sole of the foot of the user and the ground acquired by the acquisition means and a first threshold value, the gait of the user based on the information indicating the angle and a second threshold value smaller than the first threshold value; and
output information based on a result of the detection.

2. The information processing apparatus according to claim 1, wherein the at least one processor is configured to execute the instructions to acquire the information based on the sensor attached to the user at any place between the arch and the heel of the foot of the user.

3. The information processing apparatus according to claim 1, wherein the at least one processor is configured to execute the instructions to detect, in the case where the gait of the user cannot be detected based on the first threshold value when a predetermined command is received from an external device, the gait of the user based on the second threshold value.

4. The information processing apparatus according to claim 1, wherein the at least one processor is configured to execute the instructions to detect, in the case where at least one of acceleration and angular velocity measured by the sensor is equal to or greater than a threshold value and the gait of the user cannot be detected based on the first threshold value, the gait of the user based on the second threshold value.

5. The information processing apparatus according to claim 1, wherein the at least one processor is configured to execute the instructions to detect, in the case where the gait of the user cannot be detected based on the information indicating the angle sampled at a first sampling frequency and the first threshold value, the gait of the user based on the information indicating the angle sampled by a second sampling frequency higher than the first sampling frequency.

6. The information processing apparatus according to claim 1, wherein the at least one processor is configured to execute the instructions to detect, in the case where the gait of the user cannot be detected based on the first threshold value, the gait of the user based on the information indicating the angle, the second threshold value, and the acceleration in a direction in which the user walks measured by the sensor.

7. The information processing apparatus according to claim 1, wherein the at least one processor is configured to execute the instructions to determine the second threshold value based on attributes of the user.

8. The information processing apparatus according to claim 1, wherein the at least one processor is configured to execute the instructions to determine the second threshold value based on at least one of the acceleration and the angular velocity measured by the sensor.

9. The information processing apparatus according to claim 1, wherein the at least one processor is configured to execute the instructions to determine the second threshold value based on at least one of a heart rate and a skin temperature of the user.

10. An information processing method comprising:
acquiring information based on a sensor attached to a foot of a user;
detecting, when a gait of the user cannot be detected based on the acquired information indicating an angle between a sole of the foot of the user and the ground and a first threshold value, the gait of the user based on the information indicating the angle and a second threshold value lower than the first threshold value; and
outputting information based on a result of the detection.

11. A non-transitory computer-readable medium storing a program for causing an information processing apparatus to perform processes of:
acquiring information based on a sensor attached to a foot of a user;
detecting, when a gait of the user cannot be detected based on the acquired information indicating an angle between a sole of the foot of the user and the ground and a first threshold value, the gait of the user based on the information indicating the angle and a second threshold value smaller than the first threshold value; and
outputting information based on a result of the detection.
